(19) United States
(12) Patent Application Publication
(10) Pub. No.: US 2011/0105824 A1
(43) Pub. Date: May 5, 2011

(54) PARASYMPATHETICALLY INDUCED VASODILATATION METHOD AND APPARATUS

(76) Inventor: Yosef KRESPI, New York, NY (US)

(21) Appl. No.: 12/888,167

(22) Filed: Sep. 22, 2010

Related U.S. Application Data
(60) Provisional application No. 61/244,759, filed on Sep. 22, 2009.

(51) Int. Cl. A61N 5/067; A61N 5/06; A61N 2/00; A61M 37/00

(52) U.S. Cl. 600/9; 607/88; 607/89; 604/20

(57) ABSTRACT

A method and treatment device for inducing vasodilatation in a target vasculature of a patient. The method can include introducing an energy-supplying treatment instrument to a sinus cavity which provides access to a parasympathetic ganglion neurologically associated with the target vasculature. The method can also include applying non-electrical stimulant energy from the treatment instrument into the sinus cavity in a manner to stimulate the parasympathetic ganglion. The treatment device can be used to practice the method.

### CROSS-REFERENCE TO A RELATED APPLICATION

This application claims the benefit of provisional patent Application No. 61/244,759, filed on Sep. 22, 2009, the disclosure of which is incorporated by reference herein.

### STATEMENT REGARDING FEDERALLY SPONSORED RESEARCH OR DEVELOPMENT

(Not applicable.)

The present invention relates, inter alia, to a parasympathetically induced vasodilatation method and to apparatus for performing the method.

### BACKGROUND OF THE INVENTION

U.S. Pat. No. 7,117,033 to Shalev describes a method of treating a subject comprising applying an electrical current to any of a number of sites in the subject, and configuring the current so as to increase cerebral blood flow in the subject.

U.S. Pat. No. 7,190,998, also to Shalev, describes apparatus for modifying a property of a brain of a patient. The described apparatus includes one or more electrodes adapted to be applied to a sphenopalatine ganglion or other site. As described, a control unit can drive the one or more electrodes to apply to the site an electric current capable of inducing an increase in permeability of a blood brain barrier, a change in cerebral blood flow and/or inhibition of the sphenopalatine ganglion of the patient.

It would be desirable to have other methods or apparatus for induced vasodilatation via stimulation of the parasympathetic nervous system.

The foregoing description of background art may include insights, discoveries, understandings or disclosures, or associations together of disclosures, that were not known to the relevant art prior to the present invention but which were provided by the invention. Some such contributions of the invention may have been specifically pointed out herein, whereas other such contributions of the invention will be apparent from their context. Merely because a document may have been cited here, no admission is made that the field of the document, which may be quite different from that of the invention, is analogous to the field or fields of the present invention.

### BRIEF SUMMARY OF THE INVENTION

The present invention provides, in one aspect, a method of inducing vasodilatation in a target vasculature of a patient. The method can comprise a step of introducing an energy-supplying treatment instrument to a sinus cavity providing access to a parasympathetic ganglion neurologically associated with the target vasculature. The method can also comprise a further step of applying non-electrical stimulant energy from the treatment instrument into the sinus cavity in a manner to stimulate the parasympathetic ganglion. The sinus cavity can be one of the maxillary sinus cavities of a patient.

By applying suitable non-electrical energy into a sinus cavity, an associated parasympathetic ganglion can be stimulated, providing useful effects such as cerebral or other vasodilatation.

In another aspect, the invention provides a method of inducing vasodilatation in the cerebral vasculature of a patient. This method can comprise placing an energy-supplying treatment device in a sublingual location in the vicinity of a submandibular ganglion of the patient. The energy-supplying treatment device can be configured to be activatable to apply stimulant energy to stimulate the submandibular ganglion. For example, the energy-supplying treatment device can output light energy, electrical energy, ultrasound energy, magnetic energy or other suitable energy. The method can include activating the energy-supplying treatment device to stimulate the submandibular ganglion. If desired, this method can be performed without invasive surgery.

Vasodilatation induced by stimulation of a parasympathetic ganglion by a method according to the invention can be therapeutic to a victim of a stroke or the like and/or can provide time for other treatments to be employed.

In addition, or alternatively, vasodilatation induced by a method according to the invention can open the blood brain barrier to facilitate the passage of pharmacological agents for treatment of neoplasia, or for other purposes.

In another aspect, the invention provides a method of inducing vasodilatation in the cerebral vasculature of a patient. The method can comprise placing an energy-supplying treatment device in a location in the vicinity of a submandibular ganglion of the patient. The location optionally can be a sublingual location and the energy-supplying treatment device can be configured to be activatable to apply non-electrical stimulant energy to stimulate the submandibular ganglion. The method can include activating the energy-supplying treatment device to stimulate the submandibular ganglion. If desired, the energy-supplying treatment device can generate the output energy without utilizing an energy source external to the patient.

The method can include outputting light or magnetic energy from the energy-supplying treatment device. Also, if desired, the energy-supplying treatment device can generate the output energy without utilizing an energy source external to the patient. This simple method can be suitable for performance in the field, away from sophisticated medical facilities, for example by an emergency medical technician.

### BRIEF DESCRIPTION OF THE SEVERAL VIEWS OF THE DRAWING

Some embodiments of the invention, and ways of making and of using one or more embodiments of the invention, are described in detail herein and by way of example, with reference to the accompanying drawings (which are not necessarily drawn to scale with regard to any internal or external structures shown) and in which like reference characters designate like elements throughout the several views, and in which:

FIG. 1 is a cross-sectional view of a portion of the righthand side of the skull of a human subject, looking toward the corona, revealing some of the sinus cavities and showing one embodiment of a system for inducing vasodilatation according to the invention;

FIG. 2 is a view similar to FIG. 1 showing of a system for inducing vasodilatation according to another embodiment of the invention;

FIG. 3 is a coronal partial cross-sectional view of the lefthand maxillary sinus of another human subject, showing some of the soft tissues;

FIG. 4 is a schematic view of parts of one side of the cranial anatomy including the parasympathetic ganglions;

FIG. 5 is a view similar to FIG. 1 showing another embodiment of a system for inducing vasodilatation according to the invention;

FIG. 6 is a schematic view partially cut away of one side of the face; and

FIG. 7 is a cross-sectional view of an energy-supplying treatment device according to an embodiment of the invention.

### DETAILED DESCRIPTION OF THE INVENTION

Methods according to the invention can apply non-electrical stimulating energy, for example light and/or magnetic energy to one or more cranial parasympathetic ganglions to induce a useful response in the vasculature. Some aspects of the invention utilize a sinus as a treatment site for the application of stimulant energy to induce a vasodilatory or other useful response from a parasympathetic ganglion in the vicinity of the sinus. Vasodilatation of the target vasculature can be induced in any suitable manner, for example by neural impulses received from the stimulated parasympathetic ganglion.

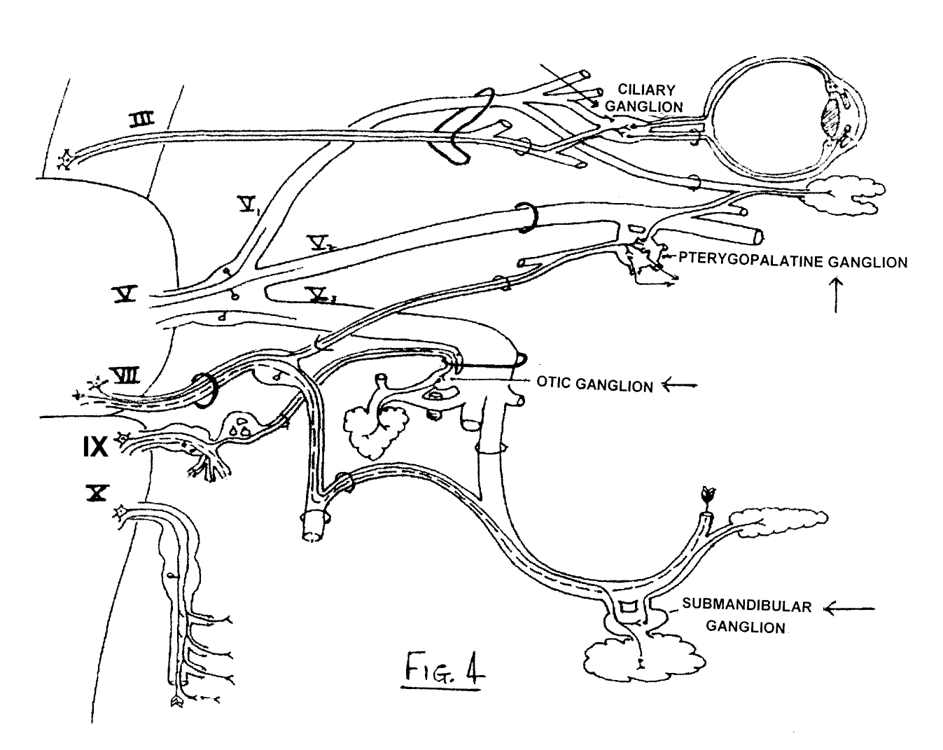

Normally, there are four parasympathetic ganglions on each side of the head, namely the sphenopalatine ganglion, the ciliary ganglion, the otic ganglion and the submandibular ganglion. Each of these ganglions is circled and marked with an arrow in FIG. 4 (the sphenopalatine ganglion is called the pterygopalatine ganglion in FIG. 4). Though it is not obvious from FIG. 4 the sphenopalatine ganglion, the ciliary ganglion and the otic ganglion are all located in the vicinity of the maxillary sinus.

In sinus treatment aspects of the invention, ganglion stimulant energy can be applied to any desired sinus that is accessible and associated with a suitable ganglion.

By applying the non-electrical stimulant energy in the maxillary sinus cavity it becomes possible to easily stimulate multiple ganglions simultaneously because several parasympathetic ganglions are usually located in the vicinity of the maxillary sinus, for example, the sphenopalatine, the ciliary or the otic ganglions. Thus, the method can be performed in a manner to stimulate one or more of these ganglions, for example all three ganglions simultaneously by applying stimulant energy in the maxillary sinus cavity. If desired, stimulant energy can also, or independently, be applied to the submandibular ganglion. Thus all four parasympathetic ganglions can be stimulated, on one or both sides of the head, if desired.

For example, stimulant energy can be applied to the back wall of the maxillary sinus, to the roof of the maxillary sinus or to both the back wall and the roof of the maxillary sinus. In some cases, such a treatment can simultaneously stimulate the sphenopalatine, the ciliary and the otic ganglions.

Methods according to the invention can be employed to treat a patient exhibiting symptoms of various cerebral and other conditions related to disorders of the cerebral vasculature, for example, at least one symptom of a condition selected from the group consisting of stroke, cerebral hemorrhage and cerebral vasospasm.

When the condition is located on one side of the head of the patient, the maxillary sinus is on the other side of the head of the patient the stimulant energy can be applied to, in, into or within the maxillary sinus on the other side of the patient. For example if a patient presents one or more symptoms associated with a stroke on the left side of the patient's head, the right side maxillary sinus can be treated to induce vasodilatation in the left cranium and possible relief of one or more stroke-related symptoms.

If desired, the stimulant energy can be applied to, in, into or within both the maxillary sinuses of the patient. It will be understood that the stimulant energy can be applied to, in, into or within one or more sinuses on one side or both sides of a patient's face.

Any suitable stimulant energy can be employed. Some embodiments of the invention employ non-electrical stimulant energy selected from the group consisting of light, ultrasound, magnetic energy and a combination of any two the foregoing energies. For example, the stimulant energy can comprise light energy having a wavelength in the range of from about 320 nm to about 1500 nm, or another suitable wavelength and, optionally, can be applied with a power output in the range of from about 2 mW to about 20 w, or another suitable power output. The light energy can be supplied in any suitable manner and can be sourced from one or more light-emitting diodes, laser diodes or other laser light or other light energy sources located externally of the patient. For example, the treatment instrument comprises an optical fiber connectable to a light source external of the patient.

The invention includes embodiments wherein light energy is output from the treatment instrument into the sinus cavity or other treatment site in a divergent manner so as to impact on target tissue over a wide angular area. For example the applied light can be diffused using a balloon, a mushroom or another suitable light diffusing device inserted into the sinus cavity, through which light is projected, for example from an optical fiber tip.

If desired, the stimulant energy can comprise a fixed or fluctuating magnetic field applied to the treatment site by a permanent magnetic device or an electromagnetic device introduced into the maxillary sinus or other sinus or treatment area. For example, a magnetic field in the range of from about 3 millitesla to about 500 millitesla, or in the range of from about 30 millitesla to about 50 millitesla, can be employed for any suitable duration, for example from about 30 seconds to one hour or from about 5 minutes to 15 minutes.

A combination of laser light energy and magnetic energy can be employed, if desired.

In another aspect of the invention the stimulant energy comprises ultrasound energy applied from an ultrasound probe introduced into the maxillary sinus or other treatment site. The ultrasound probe can be coupled to an ultrasound transducer located externally of the patient for transmission of ultrasound vibrations to the ultrasound probe.

If desired, a suitable intensity and duration for a stimulant energy treatment can be determined by routine experimentation using in vitro studies, animal studies or clinical trials.

The application of stimulant energy to the maxillary sinus or other sinus or other treatment site can have any suitable duration. In some embodiments of the invention, the duration of application of stimulant energy can be relatively short, for example in a range of from about 10 seconds to about 120 minutes, or from about 30 seconds to about 30 minutes, or from about 60 seconds to about 10 minutes. Such treatments can be repeated, as desired, for example from one to ten times in forty-eight hours at intervals of from about 30 minutes to 12 hours between treatments. Multiple treatments, for example from two to six treatments in a twenty-four period can be helpful to a stroke victim, in some cases. Alternatively, from one to ten treatments in a forty-eight hour period can be employed.

Once a single application of stimulant energy has been completed, any applicator apparatus can be withdrawn from the patient and application of energy to the patient ceases. Thus treatments can be minimally invasive and need leave nothing in the patient. However, some embodiments of the invention can employ an implanted energy emitting device, if desired.

In some aspects of the invention a medical practitioner holds, and optionally manipulates, the treatment instrument during application of the stimulant energy. The medical practitioner can partially insert the treatment instrument into the patient during the application of stimulant energy. As stated, optionally, and conveniently, the treatment instrument can be energized by an energy source external to the patient.

Methods according to the invention which apply energy to a maxillary sinus cavity can comprising introducing the treatment instrument to the maxillary sinus by piercing an access opening into the anatomy of the patient from a externally accessible anatomical location to provide access to the sinus site. Such a method can also include inserting a treatment instrument into the access opening and traversing the first access opening with the treatment instrument to introduce the treatment instrument to the maxillary sinus.

If desired, such a method can include inserting a viewing instrument into the access opening and traversing the access opening with the viewing instrument to introduce the viewing instrument to the maxillary sinus.

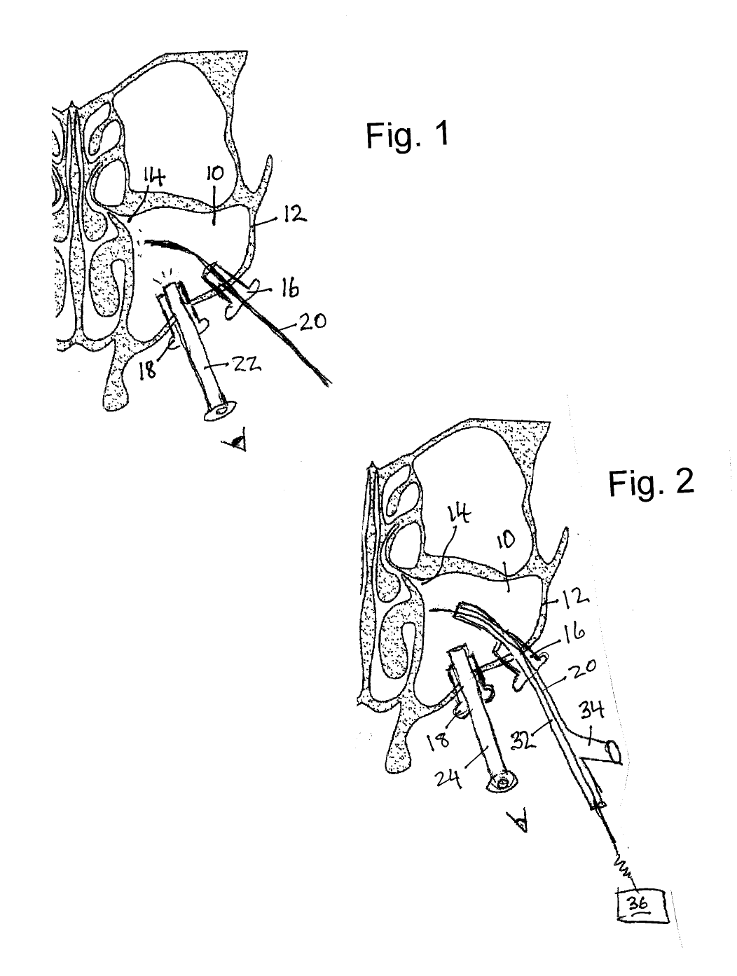

Referring to FIG. 1, some of the bony structure of the skull can be seen. This bony structure defines certain of the sinus cavities, including the maxillary antrum 10 of the maxillary sinus. The maxillary antrum 10 is further defined by the upper jawbone 12 and drains through the maxillary ostium 14

The illustrated system for inducing vasodilatation comprises a first sheath 16 which extends through a first artificial, surgically created opening in jawbone 12 which can be pierced through the canine fossa, or other suitable location above the gum line, by a trocar or another suitable piercing instrument (not shown). Viewing or monitoring of the treatment site and application of energy can be performed through this single access opening, if desired, for example by alternating instruments. Thus a viewing instrument can be introduced and withdrawn and then an energy-applying instrument can be inserted.

The invention also includes embodiments wherein two sheaths and associated openings are employed. This can be useful for viewing or monitoring the energy application as it is in progress or for other purposes, for example for treatment of a sinus condition by a method such as is described in copending provisional application No. 61/157,030 filed Mar. 3, 2009, inventor Krespi, attorney docket no. 0525497.00016, the disclosure of which is incorporated by reference herein.

Accordingly, an optional second sheath 16 can extend through a second artificial surgically created opening in jawbone 12 which can also be pierced through the canine fossa, or other suitable location above the gum line, by a trocar or another suitable piercing instrument (not shown). Desirably the first and second openings are oriented to have axial directions pointed generally toward maxillary ostium 14. In this example, the axes of the two sheaths 14 and 16, if two are employed, are spaced apart along the jawbone by a distance of about 5 to 10 mm. Usefully, each sheath can have any desired internal diameter up to about 3 or 4 mm each.

Sheath 14 and sheath 16, if the latter is employed, can be surgically inserted into the subject using any suitable procedure. For example, the subject can be given a local anesthetic, with or without sedation, prior to making the openings. Local anesthesia, if employed, can be by needle injection into the upper gum, and optionally also adjacent to the target sinus site for example by transnasal injections. For the procedure, the subject can be supine on his or her back or, can be seated in a reclinable chair such as a dental chair. If desired, the subject can be under general anesthesia during part or all of the procedure.

In the embodiment illustrated, an energy-supplying treatment instrument, for example an optical fiber 20, extends through first sheath 16 in the first opening, into maxillary antrum 10 and is shown with the tip of the laser fiber in the vicinity of maxillary ostium 14. Optical fiber 20 can be an optical fiber connecting externally with a suitable source of laser energy (not shown) for stimulating vasodilatation. A viewing instrument, for example an endoscope 22 is shown as extending through second sheath 18 in the second opening, into maxillary antrum 10 and is oriented to point toward maxillary ostium 14. Endoscope 22 can be of any suitable structure permitted by the caliber of sheath 18. For example, endoscope 22 can be rigid, if desired, can be telescopic and can provide illumination. Optical fiber 20 can be manipulated externally to apply stimulant energy from the tip of optical fiber 20 to desired surfaces or structures in the maxillary sinus or in the vicinity of the sinus. For this purpose, optical fiber 20 can be moved axially and/or rotated in sheath 16, if desired.

Some embodiments of methods of inducing vasodilatation according to the invention can be performed through a single sheath 14 and associated opening, without or without the use of a viewing instrument. If a viewing instrument, for example endoscope 22 is employed, endoscope 22 can first be inserted through sheath 14 to view the sinus cavity and obtain information helpful to the energy application step of the method. When adequate information has been gathered, endoscope 22 can be removed from sheath 14 and optical fiber 20, or other suitable energy-supplying instrument, can be introduced into sheath 14 and advanced to the maxillary sinus. Once optical fiber 20 is appropriately positioned in or close to the maxillary sinus, the energy source can be activated to supply a suitable energy dosage to maxillary sinus to stimulate the associated ganglions. If desired, the energy can be output continuously during the procedure.

Because the first and second openings are spaced apart along jawbone 12, optical fiber 20 can be moved transversely across the field of view provided through endoscope 22 which can be a valuable advantage to a surgeon or other user. Longitudinal viewing along the axis of a laser fiber or other surgical instrument while manipulating can be difficult. The possibility of lateral viewing provided by the invention can enhance depth judgment, enhance precise manipulation of the laser fiber and provide a better view of the ostium or other target site. Different locations and spacings along jawbone 12 can be selected to provide different views.

One or both of the surgical instruments, can be dual lumen instruments, if desired, enabling three or possibly more functionalities to be deployed simultaneously.

The surgical system and methodology illustrated in FIG. 2 are generally similar to the embodiment shown in FIG. 1. However, in FIG. 2, optical fiber 20 is shown inserted through a cannula 32, which can facilitate manipulation and orientation of optical fiber 20. Also cannula 32 can bring aspiration and/or irrigation to the work site via a side port 34. Optical fiber 20 is connected to a laser light source 36 of any suitable capacity and functionality, for example an energy capacity providing a power output at the tip in the range of from about 2 mW to about 20 w.

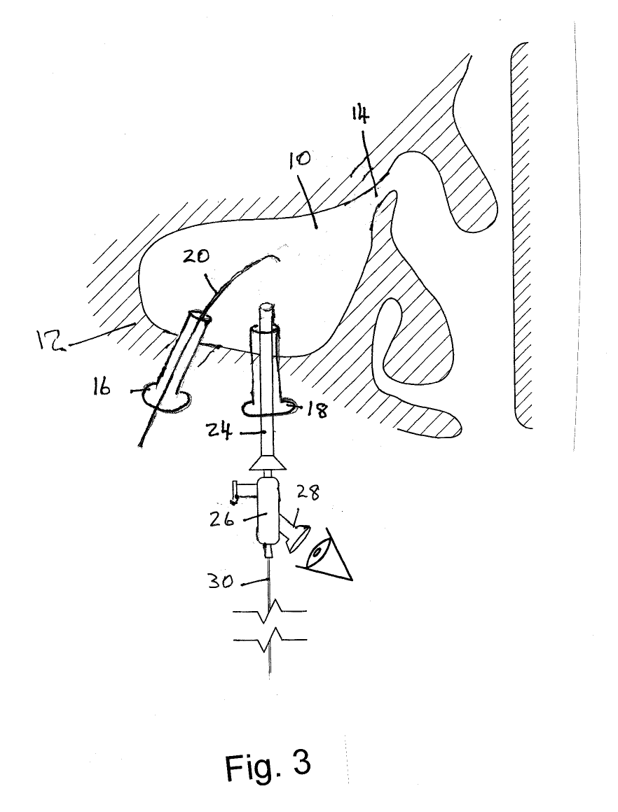

FIG. 3 shows a similar disposition of sheaths 16 and 18, optical fiber 20 and an endoscope 24 in the lefthand maxillary sinus of a subject. Endoscope 24 comprises a relatively complex optical system 26 which provides off-axis viewing through an eyepiece 28 and digital still or video imaging which can be output externally through a fiber 30.

A laser manipulator such as is described and shown in provisional application No. 61/157,030 can be employed if desired, to facilitate manipulation of optical fiber 20.

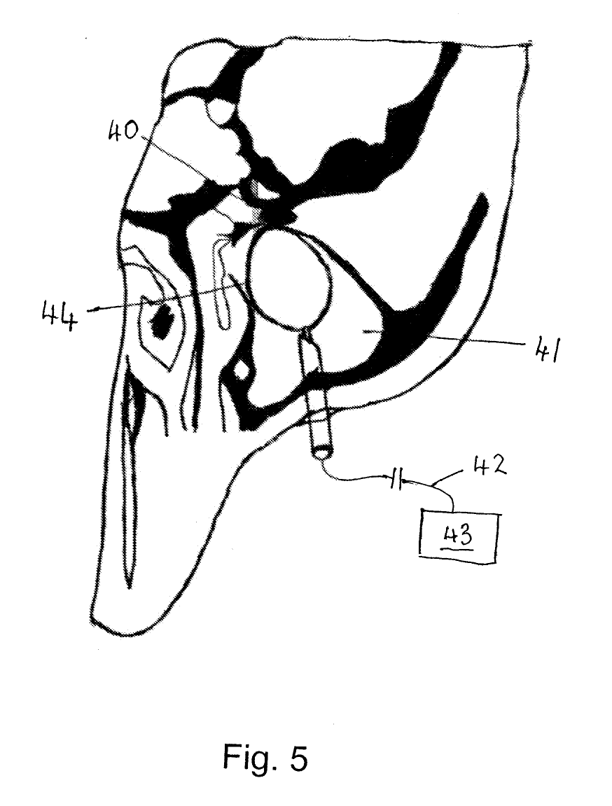

In the embodiment shown in FIG. 5, a sphenopalatine ganglion 40 can be seen behind the inward apex of a maxillary sinus 41. An optical fiber 42 is coupled to receive light energy from a light source 43, for example one or more LEDs or a laser source. Optical fiber 42 extends through a sheath 44 to a light diffuser balloon 44 located within the cavity of maxillary sinus 41. Balloon 44 can have any suitable nominal shape such as spherical, ovoid, egg-shaped or another shape and desirably is pliable to be conformable with the local anatomy at the back of the maxillary sinus cavity 41 with suitable manipulation by the medical practitioner. A light-emitting fiber tip 45 can extend from optical fiber 42 into balloon 44 and radiate light into balloon 44 and thence on to sphenopalatine ganglion 40 and on to the anatomy adjacent to sphenopalatine ganglion 40 to stimulate sphenopalatine ganglion 40, and possibly also the respective ciliary and otic ganglions.

Method embodiments of the invention also include treating patients symptomatic with headaches and/or cranial pain by applying non-electrical stimulant energy from a treatment instrument into a sinus cavity of the patient so as to stimulate the sphenopalatine ganglion. For example, relatively low energy laser-generated light can be applied to relevant nerve sheaths to ablate the nerve sheath fibers selectively using techniques such as are described herein. Demyelinization of pain nerves or nerve fibers can reduce the pain signal.

Energy wavelengths in the range of from about 650 nm to about 750 nm can be employed, for example, about 703 nm, for demyelinization, if desired. Any suitable and accessible nerve fibers can be targeted for example, the trigeminal branches of the sphenopalatine ganglion.

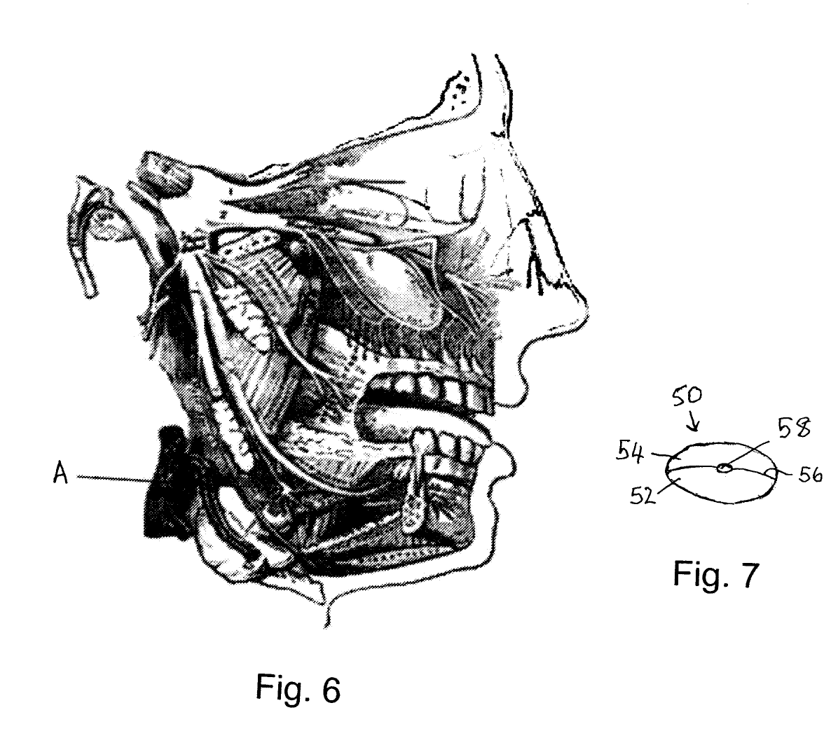

As described, the invention includes embodiments useful for stimulating the submandibular ganglion. The location of the submandibular ganglion, beneath the tongue, can be seen in FIG. 6 where it is marked with an arrow labeled A.

An energy-supplying treatment device for stimulating a submandibular ganglion of a patient can comprise an energy-generating body locatable in the vicinity of a submandibular ganglion of the patient. The location optionally can be a sublingual location. The energy-supplying treatment device can be configured to be activatable to apply non-electrical stimulant energy to stimulate the submandibular ganglion.

Desirably, the energy-generating body of the energy-supplying treatment device is configured with a suitable size, shape and external surface to fit under the tongue. One example of such an energy-supplying treatment device has an energy-generating body with a smooth surface and is shaped like a ball, a bulb, an olive, a walnut, a lozenge or a pellet or the like and has a largest dimension in the range of from about 5 to about 60 mm. The device can include a magnet, for example a donut magnet or a light source, optionally a light-emitting diode (“LED” herein), or a cluster of LEDs. If a light source is employed, the device can also include an energy source to power the light source, for example a battery. The device can include both a magnet and a light source, if desired.

One embodiment of energy-supplying treatment device comprises a crushable light comprising one or more LEDs which is activated by crushing and then continuously emits a burst of light until the energy supply dissipates after a predetermined time interval, for example from one to four hours. Another embodiment comprises a crushable light bulb, ball, nut or lozenge having transparent outer wall or wall portion through which light is emitted after the device is crushed.

Such a device can be suitable for emergency use by a technician or other user in the field and can, in some cases, provide useful immediate treatment for a stroke victim, pending availability of, or transport of the victim to more sophisticated treatments. If desired, cotton wadding, ties or the like can be employed to hold the energy-supplying treatment device in place under the tongue.

As an alternative to sublingual location, if desired, the energy-supplying treatment device can be located externally beneath the chin in the vicinity of the submandibular ganglion to apply stimulant energy to the submandibular ganglion, and can be held in place by adhesive tape, ties or another suitable device.

Referring to FIG. 7, the embodiment of energy-supplying treatment device shown comprises an energy-generating body 50 having a transparent lower portion 52 and an opaque upper portion 54 divided by a light reflector 56 which is concave on the downward light-transmitting side of the device. An LED 58 is mounted approximately centrally of the device. The upper opaque portion includes a power supply, for example a battery, and a switch to activate and deactivate the light. This device can be located under the tongue or taped externally beneath the chin with transparent portion 52 facing the submandibular ganglion.

Thus, the invention provides, inter alia, methods and devices for inducing vasodilatation of target vasculature which can be easily effected with modest or no surgery and without requiring use of an implant.

Patient. Useful embodiments of the invention can be applied to, or adapted, for a wide variety of human patients of either gender, and of various ages ethnicities and health categories. Usefully, the patient can be a patient presenting one or more symptoms of a condition treatable by vasodilatation of the target vasculature.

If desired, the methods, principles, systems, instruments or apparatus of the invention can also be applied to treatment of one or more non-human mammals including for example, horses, cattle, sheep and other husbanded animals, pets such as dogs and cats, laboratory animals for example mice, rats, rabbits, apes, monkeys and other primates, animals employed for sports, entertainment, law enforcement, draft usage, zoological or other purposes.

Disclosures Incorporated. The entire disclosure of each and every United States patent and patent application, each foreign and international patent publication, of each other publication and of each unpublished patent application that is specifically referenced in this specification is hereby incorporated by reference herein, in its entirety. Should there appear to be conflict between the meaning of a term employed in the description of the invention in this specification and with the usage in material incorporated by reference from another document, the meaning as used herein is intended to prevail.

The foregoing detailed description is to be read in light of and in combination with the preceding background and invention summary descriptions wherein partial or complete information regarding the best mode of practicing the invention, or regarding modifications, alternatives or useful embodiments of the invention may also be set forth or suggested, as will be apparent to one skilled in the art.

The terms “include,” “have,” “has,” and “contain,” and their various grammatical forms, are to be understood as being open-ended and not to exclude additional, unrecited elements or method steps.

Throughout the description, where compositions instruments, devices apparatus, systems, or processes are described as having, including, or comprising specific components or elements, or in the case of processes, specific steps, it is contemplated that compositions instruments, devices apparatus, systems, or processes according to the present invention can also consist essentially of, or consist only of, the recited components, elements or steps.

In the application, where an element or component is said to be included in and/or selected from a list or group of recited elements or components, it should be understood that the element or component can be any one of the recited elements or components or can be selected from a group consisting of two or more of the recited elements or components.

The use of the singular herein is intended to include the plural (and vice versa) unless the context indicates otherwise. In addition, where the term “about” is used before a quantitative value, the specific quantitative value itself is intended to be included, unless specifically stated otherwise.

With regard to processes, it is to be understood that the order of steps or order for performing certain actions is immaterial so long as the described process remains operable. Moreover, two or more steps or actions may be conducted simultaneously, unless the context indicates otherwise. In addition, any proportions recited herein are to be understood to be proportions by weight, based upon the weight of the relevant composition, unless the context indicates otherwise.

The description of the invention is to be understood as including combinations of the various elements of the invention, and of their disclosed or suggested alternatives, including alternatives disclosed, implied or suggested in any one or more of the various methods, products, compositions, systems, apparatus, instruments, aspects, embodiments, examples described in the specification or drawings, if any, and to include any other written or illustrated combination or grouping of elements of the invention or of the possible practice of the invention, except for groups or combinations of elements that are incompatible with, or contrary to the purposes of the invention, as will be or become apparent to a person of ordinary skill.

The present invention includes the examples and embodiments described herein and other specific forms that embody the spirit or essential characteristics of the invention or of the respective described example or embodiment. The foregoing examples and embodiments are in all respects intended to be illustrative of the invention described herein. It is to be understood that many and various modifications of the invention, or of an example or embodiment of the invention described herein will be apparent to those of ordinary skill in the relevant art, or may become apparent as the art develops, in the light of the foregoing description. Such modifications are contemplated as being within the spirit and scope of the invention or inventions disclosed herein.

1. A method of inducing vasodilatation in a target vasculature of a patient, the method comprising:
introducing an energy-supplying treatment instrument to a sinus cavity, the sinus cavity providing access to a parasympathetic ganglion neurologically associated with the target vasculature; and
applying non-electrical stimulant energy from the treatment instrument into the sinus cavity in a manner to stimulate the parasympathetic ganglion.

2. A method according to claim 1 wherein the sinus cavity is a maxillary sinus cavity.

3. A method according to claim 2 comprising applying the stimulant energy in the maxillary sinus cavity in a manner to stimulate one or more of the sphenopalatine, the ciliary or the otic ganglions.

4. A method according to claim 2 comprising applying the stimulant energy in the maxillary sinus cavity in a manner to stimulate the sphenopalatine, the ciliary and the otic ganglions.

5. A method according to claim 2 wherein the patient exhibits at least one symptom of a condition selected from the group consisting of stroke, cerebral hemorrhage and cerebral vasospasm.

6. A method according to claim 5 wherein the condition is located on one side of the head of the patient and the maxillary sinus is on the other side of the head of the patient.

7. A method according to claim 1 wherein the stimulant energy is selected from the group consisting of light, light output from one or more light-emitting diodes, magnetic energy and a combination of any two of the foregoing energies.

8. A method according to claim 1 wherein the stimulant energy comprises laser light energy having a wavelength in the range of from about 320 nm to about 1500 nm and, optionally, magnetic energy.

9. A method according to claim 1 comprising applying the stimulant energy continuously to the sinus cavity for a duration of from 30 seconds to 60 minutes.

10. A method according to claim 9 wherein the stimulant energy application is repeated from one to ten times in forty-eight hours.

11. A method according to claim 1 comprising a medical practitioner holding the treatment instrument during application of the stimulant energy and the medical practitioner partially inserting the treatment instrument into the patient during the application of stimulant energy wherein, optionally, the treatment instrument is energized by an energy source external to the patient.

12. A method according to claim 111 wherein the stimulant energy comprises light and the treatment instrument comprises an optical fiber connectable to a light source located externally of the patient.

13. A method according to claim 1 comprising sourcing the energy externally of the patient and transmitting the energy from the source to the treatment site.

14. A method according to claim 1 wherein the patient is in need of treatment for a condition treatable by vasodilatation of the target vasculature and the sinus cavity is a maxillary sinus cavity, the method comprising introducing the treatment instrument to the maxillary sinus by piercing an access opening into the anatomy of the patient from a externally accessible anatomical location to provide access to the sinus site, inserting a treatment instrument into the access opening and traversing the first access opening with the treatment instrument to introduce the treatment instrument to the maxillary sinus.

15. A method according to claim 14 comprising inserting a viewing instrument into the access opening and traversing the access opening with the viewing instrument to introduce the viewing instrument to the maxillary sinus.

16. A method of inducing vasodilatation in the cerebral vasculature of a patient, the method comprising:
placing an energy-supplying treatment device in a location in the vicinity of a submandibular ganglion of the patient, the location optionally being a sublingual location, wherein the energy-supplying treatment device is configured to be activatable to apply stimulant energy to stimulate the submandibular ganglion; and
activating the energy-supplying treatment device to stimulate the submandibular ganglion.

17. A method of drug delivery comprising opening the blood-brain barrier by performing a method according to claim 1.

18. A method of treating a stroke victim comprising performing a method according to claim 1.

19. An energy-supplying treatment device for stimulating a submandibular ganglion of a patient, the treatment device comprising an energy-generating body locatable in the vicinity of a submandibular ganglion of the patient, the location optionally being a sublingual location, wherein the energy-supplying treatment device is configured to be activatable to apply stimulant energy to stimulate the submandibular ganglion.

20. An apparatus, device or system for performing a method according to claim 1.
